(19) United States
(12) Patent Application Publication
(10) Pub. No.: US 2020/0268628 A1
(43) Pub. Date: Aug. 27, 2020

(54) OIL-IN-WATER EMULSION COMPOSITION COMPRISING ETHER OIL

(71) Applicant: L'OREAL, Paris (FR)

(72) Inventors: Fan HU, Hangzhou, Zhejiang (CN); Hiroaki KAGA, Kawasaki-shi (JP); Bradford PISTORIO, Clark, NJ (US)

(21) Appl. No.: 16/634,799

(22) PCT Filed: Aug. 10, 2018
(86) PCT No.: PCT/JP2018/030455

§ 371 Date: Jan. 28, 2020

(30) Foreign Application Priority Data
Aug. 29, 2017 (JP) 2017-164105

(51) Int. Cl. A61K 8/39; A61K 8/06; A61K 8/92; A61K 8/86; A61K 8/31; A61Q 1/14

(52) U.S. Cl. CPC A61K 8/39; A61K 8/062; A61K 8/92; A61K 8/86; A61K 2800/524; A61Q 1/14; A61K 2800/21; A61K 2800/10; A61K 8/31

(57) ABSTRACT

One embodiment of the invention relates to a composition in the form of an oil-in-water emulsion. The composition contains (a) at least one ether oil; (b) at least one nonionic surfactant; and (c) water. The amount of the (c) water in the composition is 70% by weight or more, preferably 80% by weight or more, and more preferably 90% by weight or more, relative to the total weight of the composition. The oil droplet size in the composition is less than 100 nm, preferably less than 90 nm, and more preferably less than 80 nm. The composition according to this embodiment can have a good cleansing ability and a transparent or slightly translucent appearance.

### TECHNICAL FIELD

The present invention relates to a composition in the form of an oil-in-water emulsion, preferably a cosmetic composition, and more preferably a cleansing composition.

### BACKGROUND ART

Cleansing the skin is very important when caring for the face. The cleansing must be as effective as possible because greasy residues, such as excess sebum, the remnants of cosmetic products used daily, and make-up products, in particular waterproof products, tend to accumulate in skin folds, and block the pores of the skin resulting in the appearance of spots.

Several types of skin cleansing products, for example, rinsable cleansing anhydrous oils and gels, and foaming creams, lotions and gels, are known.

Rinsable anhydrous oils and gels have a cleansing action mainly by virtue of oils present in these formulations. These oils make it possible to dissolve fatty residues and disperse make-up pigments. These products are effective and well tolerated. However, they exhibit the disadvantages of being heavy, of not foaming, and of not conferring a feeling of freshness on application all of which are disadvantageous from a cosmetic viewpoint.

On the other hand, foaming creams, lotions and gels without oils have a cleansing action mainly by virtue of the surfactants therein, which suspend the fatty residues and the pigments of, for example, the make-up products. They are effective and pleasant to use because they foam and are easy to remove.

### DISCLOSURE OF INVENTION

Recently, cleansing products in particular facial cleansing compositions are expected to provide not only cleansing effects but also a good appearance such as transparency or slight translucency and a good feeling on the skin after use such as a refreshing, smoothing and moisturizing sensation.

In order to provide a good feeling such as a refreshing sensation on the skin after use, it may be preferable to use a substantial amount of water in cleansing products. However, the use of a substantial amount of water substantially reduces the proportion of oil in the cleansing products and typically deteriorates the cleansing ability of the cleansing products.

An objective of the present invention is to provide a composition which has a good cleansing ability and a transparent or slightly translucent appearance even though the composition includes a substantial amount of water.

The above objective of the present invention can be achieved by a composition in the form of an oil-in-water emulsion, comprising:
(a) at least one ether oil;
(b) at least one nonionic surfactant; and
(c) water,
   wherein
   the amount of the (c) water in the composition is 70% by weight or more, preferably 80% by weight or more, and more preferably 90% by weight or more relative to the total weight of the composition, and
   the oil droplet size in the composition is less than 100 nm, preferably less than 90 nm, and more preferably less than 80 nm.

The (a) ether oil may be selected from the group consisting of dicaprylyl ether, dicapryl ether, dilauryl ether, diisostearyl ether, dioctyl ether, nonyl phenyl ether, dodecyl dimethylbutyl ether, cetyl dimethylbutyl ether, cetyl isobutyl ether, and mixtures thereof.

The amount of the (a) ether oil(s) in the composition may range from 0.01 to 20% by weight, preferably from 0.1 to 10% by weight, and more preferably from 0.5 to 5% by weight relative to the total weight of the composition.

The (b) nonionic surfactant may have an HLB value of from 8.0 to 14.0, preferably from 9.0 to 13.5, and more preferably from 10.0 to 13.0.

The (b) nonionic surfactant may be chosen from:
(1) surfactants chosen from polyglyceryl fatty acid esters, polyoxyalkylenated alkyl glycerides, and polyoxyalkylenated fatty ethers;
(2) mixed esters of fatty acid or of fatty alcohol, of carboxylic acid and of glycerol;
(3) fatty acid esters of sugars and fatty alcohol ethers of sugars;
(4) surfactants chosen from fatty esters of sorbitan and oxyalkylenated fatty esters of sorbitan, and oxyalkylenated fatty esters;
(5) block copolymers of ethylene oxide (A) and of propylene oxide (B),
(6) polyoxyethylenated (1-40 EO) and polyoxypropylenated (1-30 PO) alkyl (C₁₆-C₃₀) ethers,
(7) silicone surfactants, and
(8) mixtures thereof.

The (b) nonionic surfactant may be chosen from:
polyglyceryl fatty acid esters of at least one, preferably one, fatty acid comprising at least one saturated or unsaturated, linear or branched C₈-C₂₂ hydrocarbon group such as C₈-C₂₂ alkyl or alkenyl group, preferably C₈-C₁₈ alkyl or alkenyl group, and more preferably C₈-C₁₂ alkyl or alkenyl group, and of 2 to 12 glycerols, preferably 2 to 10 glycerols and more preferably 2 to 8 glycerols;
polyoxyethylenated alkyl glycerides such as polyethylene glycol derivatives of a mixture of mono-, di- and tri-glycerides of caprylic and capric acids (preferably 2 to 30 ethylene oxide units, more preferably 2 to 20 ethylene oxide units, and even more preferably 2 to 10 ethylene oxide units);
polyoxyethylenated fatty ethers of at least one, preferably one, fatty alcohol comprising at least one saturated or unsaturated, linear or branched C₈-C₂₂ hydrocarbon group such as C₈-C₂₂ alkyl or alkenyl group, preferably C₈-C₁₈ alkyl or alkenyl group, and more preferably C₈-C₁₂ alkyl or alkenyl group, and of 2 to 60 ethylene oxides, preferably from 2 to 30 ethylene oxides, and more preferably from 2 to 10 ethylene oxides; and
mixtures thereof.

The amount of the (b) nonionic surfactant(s) in the composition may range from 0.1 to 30% by weight, preferably from 0.5 to 20% by weight, and more preferably from 1 to 10% by weight relative to the total weight of the composition.

The weight ratio of the (b) nonionic surfactant(s) to the (a) ether oil(s) may be from 1:1 to 3:1, preferably from 1:1 to 2.67:1, and more preferably from 1:1 to 2:1.

The composition may further comprise (d) at least one additional oil.

The (d) additional oil may be selected from non-polar oils, preferably hydrocarbon oils, and more preferably volatile hydrocarbon oils.

The amount of the (d) additional oil(s) in the composition may range from 0.01 to 20% by weight, preferably from 0.1 to 10% by weight, and more preferably from 0.5 to 5% by weight relative to the total weight of the composition.

The total amount of the (a) ether oil(s) and the (d) additional oil(s) in the composition may range from 0.01 to 20% by weight, preferably from 0.1 to 10% by weight, and more preferably from 0.5 to 5% by weight relative to the total weight of the composition.

The composition according to the present invention may be used as is or may be used for a cleansing product, preferably a make-up cleansing product for skin, and more preferably a make-up cleansing product for body and/or face.

The present invention also relates to a process for cleansing a keratin substance such as skin, hair, mucous membranes, nails, eyelashes, eyebrows, and scalp, comprising the step of applying the composition according to the present invention to the keratin substance.

The present invention also relates to a use of (a) at least one ether oil in a composition, in the form of an oil-in-water emulsion, comprising (b) at least one nonionic surfactant and (c) water, wherein the amount of the (c) water in the composition is 70% by weight or more, preferably 80% by weight or more, and more preferably 90% by weight or more relative to the total weight of the composition, in order to make the oil droplet size in the composition less than 100 nm, preferably less than 90 nm, and more preferably less than 80 nm, and/or in order to enhance the cleansing ability of the composition.

### BRIEF DESCRIPTION OF DRAWING

FIG. 1 shows a graph showing the makeup removability of the compositions according to Examples A-F and Comparative Examples 1-4.

### BEST MODE FOR CARRYING OUT THE INVENTION

After diligent research, the inventors have discovered that it is possible to provide a composition which can have a good cleansing ability and a transparent or slightly translucent appearance even though the composition includes a substantial amount of water.

Specifically, it was discovered that the use of at least one ether oil in a composition, in the form of an oil-in-water emulsion, comprising at least one nonionic surfactant and water in an amount of 70% by weight or more, preferably 80% by weight or more, and more preferably 90% by weight or more relative to the total weight of the composition can make the oil droplet size in the composition less than 100 nm, preferably less than 90 nm, and more preferably less than 80 nm, thereby giving the composition a transparent or slightly translucent appearance and a good cleansing ability.

Thus, the composition according to the present invention is a composition in the form of an oil-in-water emulsion, comprising:
(a) at least one ether oil;
(b) at least one nonionic surfactant; and
(c) water,
   wherein
   the amount of the (c) water in the composition is 70% by weight or more, preferably 80% by weight or more, and more preferably 90% by weight or more relative to the total weight of the composition, and
   the oil droplet size in the composition is less than 100 nm, preferably less than 90 nm, and more preferably less than 80 nm.

The composition according to the present invention can have a good cleansing ability and a transparent or slightly translucent appearance.

Since the composition according to the present invention includes (a) ether oil(s), as well as (b) nonionic surfactant(s), it can have a cleansing ability.

On the other hand, since the oil droplet size (particle diameter) in the composition according to the present invention is very small, the composition according to the present invention can be in the form of a nano- or micro-emulsion, and therefore, it can be transparent or slightly translucent (not opaque).

Also, as the composition according to the present invention includes a substantial amount of water, which forms the outer phase of the composition in the form of an oil-in-water emulsion, the composition according to the present invention can provide good feeling on the skin after use, such as refreshing, smoothing and moisturizing sensation.

Hereinafter, the composition, process and use, according to the present invention will be explained in a more detailed manner.

### [Composition]

### (Ether Oil)

The composition according to the present invention comprises (1) at least one ether oil. A single type of ether oil may be used, but two or more different types of ether oils may be used in combination.

Here, “oil” means a fatty compound or substance that is in the form of a liquid or a paste (non-solid) at room temperature (25° C.) and under atmospheric pressure (760 mmHg).

The (a) ether oil may be volatile or non-volatile, preferably non-volatile.

It may be preferable to use, as the (a) ether oil, dialkyl ethers such as those represented by the following formula:

R¹—O—R²

wherein
each of R¹ and R² independently denotes a linear, branched or cyclic C₄₋₂₄ alkyl group, preferably C₆₋₁₈ alkyl group, and more preferably C₈₋₁₂ alkyl group. It may be preferable that R¹ and R² are the same.

As the linear alkyl group, mention may be made of a butyl group, a pentyl group, a hexyl group, a heptyl group, an octyl group, a nonyl group, a decyl group, an undecyl group, a dodecyl group, tridecyl group, a tetradecyl group, a pentadecyl group, a hexadecyl group, a heptadecyl group, an octadecyl group, a nonadecyl group, an eicosyl group, a behenyl group, a docosyl group, a tricosyl group, and an tetracosyl group.

As the branched alkyl group, mention may be made of a 1-methylpropyl group, 2-methylpropyl group, a t-butyl group, a 1,1-dimethylpropyl group, a 3-methylhexyl group, a 5-methylhexyl group, an 1-ethylhexyl group, an 2-ethylhexyl group, a 1-butylpentyl group, a 5-methyloctyl group, an 1-ethylhexyl group, an 2-ethylhexyl group, a 1-butylpentyl group, a 5-methyloctyl group, a 2-butyloctyl group, an isotridecyl group, a 2-pentylnonyl group, a 2-hexyldecyl group, an isostearyl group, a 2-heptylundecyl group, an 2-octyldodecyl group, a 1,3-dimethylbutyl group, a 1-(1-methylethyl)-2-methylpropyl group, a 1,1,3,3-tetramethylbutyl group, a 3,5,5-trimethylhexyl group, a 1-(2-methylpropyl)-3-methylbutyl group, a 3,7-dimethyloctyl group, and a 2-(1,3,3-trimethylbutyl)-5,7,7-trimethyloctyl group.

As the cyclic alkyl group, mention may be made of a cyclohexyl group, a 3-methylcyclohexyl group, and a 3,3,5-trimethylcyclohexyl group.

It may be preferable that the (a) ether oil be selected from the group consisting of dicaprylyl ether, dicapryl ether, dilauryl ether, diisostearyl ether, dioctyl ether, nonyl phenyl ether, dodecyl dimethylbutyl ether, cetyl dimethylbutyl ether, cetyl isobutyl ether, and mixtures thereof.

It may be more preferable that the (a) ether oil be selected from the group consisting of dicaprylyl ether, dicapryl ether, dilauryl ether, diisostearyl ether, dioctyl ether, and mixtures thereof.

The amount of the (a) ether oil(s) in the composition according to the present invention may be 0.01% by weight or more, preferably 0.1% by weight or more, and more preferably 0.5% by weight or more relative to the total weight of the composition. It may be even more preferable that the amount of the (a) ether oil(s) in the composition according to the present invention be 1% by weight or more relative to the total weight of the composition.

On the other hand, the amount of the (a) ether oil(s) in the composition according to the present invention may be 20% by weight or less, preferably 10% by weight or less, and more preferably 5% by weight or less relative to the total weight of the composition. It may be even more preferable that the amount of the (a) ether oil(s) in the composition according to the present invention be 3% by weight or less relative to the total weight of the composition.

The amount of the (a) ether oil(s) in the composition according to the present invention may range from 0.01 to 20% by weight, preferably from 0.1 to 10% by weight, more preferably from 0.5 to 5% by weight relative to the total weight of the composition. It may be even more preferable that the amount of the (a) ether oil(s) in the composition according to the present invention be from 1% to 3% by weight relative to the total weight of the composition.

### (Nonionic Surfactant)

The composition according to the present invention comprises (b) at least one nonionic surfactant. A single type of the specific nonionic surfactant may be used, but two or more different types of the specific nonionic surfactant may be used in combination.

The nonionic surfactant may have an HLB (Hydrophilic Lipophilic Balance) value of from 8.0 to 14.0, preferably from 9.0 to 13.5, and more preferably from 10.0 to 13.0. If two or more nonionic surfactants are used, the HLB value is determined by the weight average of the HLB values of all the nonionic surfactants.

The (b) nonionic surfactant may be chosen from:
(1) surfactants chosen from polyglyceryl fatty acid esters, polyoxyalkylenated alkyl glycerides, and polyoxyalkylenated fatty ethers;
(2) mixed esters of fatty acid or of fatty alcohol, of carboxylic acid and of glycerol;
(3) fatty acid esters of sugars and fatty alcohol ethers of sugars;
(4) surfactants chosen from fatty esters of sorbitan and oxyalkylenated fatty esters of sorbitan, and oxyalkylenated fatty esters;
(5) block copolymers of ethylene oxide (A) and of propylene oxide (B),
(6) polyoxyethylenated (1-40 EO) and polyoxypropylenated (1-30 PO) alkyl (C₁₆-C₃₀) ethers,
(7) silicone surfactants, and
(8) mixtures thereof.

The surfactant (1) may be a fluid at a temperature of less than or equal to 45° C.

The surfactant (1) may be in particular:
polyglyceryl fatty acid esters of at least one, preferably one, fatty acid comprising at least one saturated or unsaturated, linear or branched C₈-C₂₂ hydrocarbon group such as C₈-C₂₂ alkyl or alkenyl group, preferably C₈-C₁₈ alkyl or alkenyl group, and more preferably C₈-C₁₂ alkyl or alkenyl group, and of 2-12 glycerols, preferably 2-10 glycerols and more preferably 2-8 glycerols;
polyoxyethylenated (PEGylated) alkyl glycerides such as polyethylene glycol derivatives of a mixture of mono-, di- and tri-glycerides of caprylic and capric acids (preferably 2 to 30 ethylene oxide units, more preferably 2 to 20 ethylene oxide units, and even more preferably 2 to 10 ethylene oxide units), e.g., PEG-6 Caprylic/Capric Glycerides, PEG-7 Caprylic/Capric Glycerides, and PEG-7 glyceryl cocoate;
polyoxyethylenated fatty ethers of at least one, preferably one, fatty alcohol comprising at least one saturated or unsaturated, linear or branched C₈-C₂₂ hydrocarbon group such as C₈-C₂₂ alkyl or alkenyl group, preferably C₈-C₁₈ alkyl or alkenyl group, and more preferably C₈-C₁₂ alkyl or alkenyl group, and of 2 to 60 ethylene oxides, preferably from 2 to 30 ethylene oxides, and more preferably from 2 to 10 ethylene oxides; and
mixtures thereof.

If the (b) nonionic surfactant is selected from the above polyglyceryl fatty acid esters, polyoxyalkylenated alkyl glycerides, polyoxyalkylenated fatty ethers, and mixtures thereof, a flexible interfacial film or layer can be formed between the (a) ether oil and (c) water. The flexible interfacial film or layer can effectively reduce the interfacial tension between the (a) ether oil and (c) water, and this can make it easier to obtain oil drolets with a size of less than 100 nm. Furthermore, the flexible interfacial film or later can be mild to skin and eyelids as well as eyes.

It is preferable that the (b) nonionic surfactant is a mixture of at least two selected from the above polyglyceryl fatty acid esters, polyoxyethylenated alkyl glycerides, and polyoxyethylenated fatty ethers.

It is preferable that the polyglyceryl fatty acid ester have a polyglycerol moiety derived from 2 to 10 glycerols, more preferably from 2 to 8 glycerols, and further more preferably 4 to 6 glycerols.

The polyglyceryl fatty acid ester may be chosen from the mono, di and tri esters of saturated or unsaturated acid, preferably saturated acid, including 8 to 22 carbon atoms, preferably 8 to 18 carbon atoms, and more preferably 8 to 12 carbon atoms, such as caprylic acid, capric acid, lauric acid, oleic acid, stearic acid, isostearic acid, and myristic acid.

The polyglyceryl fatty acid ester may be selected from the group consisting of PG2 caprate, PG2 dicaprate, PG2 tricaprate, PG2 caprylate, PG2 dicaprylate, PG2 tricaprylate, PG2 laurate, PG2 dilaurate, PG2 trilaurate, PG2 myristate, PG2 dimyristate, PG2 trimyristate, PG2 stearate, PG2 distearate, PG2 tristearate, PG2 isostearate, PG2 diisostearate, PG2 triisostearate, PG2 oleate, PG2 dioleate, PG2 trioleare, PG3 caprate, PG3 dicaprate, PG3 tricaprate, PG3 caprylate, PG3 dicaprylate, PG3 tricaprylate, PG3 laurate, PG3 dilaurate, PG3 trilaurate, PG3 myristate, PG3 dimyristate, PG3 trimyristate, PG3 stearate, PG3 distearate, PG3 tristearate, PG3 isostearate, PG3 diisostearate, PG3 triisostearate, PG3 oleate, PG3 dioleate, PG3 trioleare, PG4 caprate, PG4 dicaprate, PG4 tricaprate, PG4 caprylate, PG4 dicaprylate, PG4 tricaprylate, PG4 laurate, PG4 dilaurate, PG4 trilaurate, PG4 myristate, PG4 dimyristate, PG4 trimyristate, PG4 stearate, PG4 distearate, PG4 tristearate, PG4 isostearate, PG4 diisostearate, PG4 triisostearate, PG4 oleate, PG4 dioleate, PG4 trioleare, PG5 caprate, PG5 dicaprate, PG5 tricaprate, PG5 caprylate, PG5 dicaprylate, PG5 tricaprylate, PG5 laurate, PG5 dilaurate, PG5 trilaurate, PG5 myristate, PG5 dimyristate, PG5 trimyristate, PG5 stearate, PG5 distearate, PG5 tristearate, PG5 isostearate, PG5 diisostearate, PG5 triisostearate, PG5 oleate, PG5 dioleate, PG5 trioleare, PG6 caprate, PG6 dicaprate, PG6 tricaprate, PG6 caprylate, PG6 dicaprylate, PG6 tricaprylate, PG6 laurate, PG6 dilaurate, PG6 trilaurate, PG6 myristate, PG6 dimyristate, PG6 trimyristate, PG6 stearate, PG6 distearate, PG6 tristearate, PG6 isostearate, PG6 diisostearate, PG6 triisostearate, PG6 oleate, PG6 dioleate, PG6 trioleare, PG10 caprate, PG10 dicaprate, PG10 tricaprate, PG10 caprylate, PG10 dicaprylate, PG10 tricaprylate, PG10 laurate, PG10 dilaurate, PG10 trilaurate, PG10 myristate, PG10 dimyristate, PG10 trimyristate, PG10 stearate, PG10 distearate, PG10 tristearate, PG10 isostearate, PG10 diisostearate, PG10 triisostearate, PG10 oleate, PG10 dioleate, and PG10 trioleare.

The polyoxyalkylenated fatty ethers, preferably polyoxyethylenated fatty ethers, may comprise from 2 to 60 ethylene oxide units, preferably from 2 to 30 ethylene oxide units, and more preferably from 2 to 10 ethylene oxide units. The fatty chain of the ethers may be chosen in particular from lauryl, behenyl, arachidyl, stearyl and cetyl units, and mixtures thereof, such as cetearyl. Examples of ethoxylated fatty ethers which may be mentioned are lauryl alcohol ethers comprising 2, 3, 4, and 5 ethylene oxide units (CTFA names: Laureth-2, Laureth-3, Laureth-4, and Laureth-5, such as the products sold under the names Nikkol BL-2 by the company Nikko Chemicals, Emalex 703 by the company Nihon Emulsion Co., Ltd, Nikkol BL-4 by the company Nikko Chemicals, and EMALEX 705 by the company Nihon Emulsion Co., Ltd.

The (2) mixed esters of fatty acids, or of fatty alcohol, of carboxylic acid and of glycerol, which can be used as the above nonionic surfactant, may be chosen in particular from the group comprising mixed esters of fatty acid or of fatty alcohol with an alkyl or alkenyl chain containing from 8 to 22 carbon atoms, preferably from 8 to 18 carbon atoms, and more preferably from 8 to 12 carbon atoms, and of α-hydroxy acid and/or of succinic acid, with glycerol. The α-hydroxy acid may be, for example, citric acid, lactic acid, glycolic acid or malic acid, and mixtures thereof.

The alkyl chain of the fatty acids or alcohols from which are derived the mixed esters which can be used in the nanoemulsion of the invention may be linear or branched, and saturated or unsaturated. They may especially be stearate, isostearate, linoleate, oleate, behenate, arachidonate, palmitate, myristate, laurate, caprate, isostearyl, stearyl, linoleyl, oleyl, behenyl, myristyl, lauryl or capryl chains, and mixtures thereof.

As examples of mixed esters which can be used in the nanoemulsion of the invention, mention may be made of the mixed ester of glycerol and of the mixture of citric acid, lactic acid, linoleic acid and oleic acid (CTFA name: Glyceryl citrate/lactate/linoleate/oleate) sold by the company Hills under the name Imwitor 375; the mixed ester of succinic acid and of isostearyl alcohol with glycerol (CTFA name: Isostearyl diglyceryl succinate) sold by the company Hills under the name Imwitor 780 K; the mixed ester of citric acid and of stearic acid with glycerol (CTFA name: Glyceryl stearate citrate) sold by the company Hills under the name Imwitor 370; the mixed ester of lactic acid and of stearic acid with glycerol (CTFA name: Glyceryl stearate lactate) sold by the company Danisco under the name Lactodan B30 or Rylo LA30.

The (3) fatty acid esters of sugars, which can be used as the above nonionic surfactant, may be chosen in particular from the group comprising esters or mixtures of esters of C₈-C₂₂ fatty acid and of sucrose, of maltose, of glucose or of fructose, and esters or mixtures of esters of C₁₄-C₂₂ fatty acid and of methylglucose.

The C₈-C₂₂ or C₁₄-C₂₂ fatty acids forming the fatty unit of the esters which can be used in the present invention comprise a saturated or unsaturated linear alkyl or alkenyl chain containing, respectively, from 8 to 22 or from 14 to 22 carbon atoms. The fatty unit of the esters may be chosen in particular from stearates, behenates, arachidonates, palmitates, myristates, laurates and caprates, and mixtures thereof. Stearates are preferably used.

As examples of esters or mixtures of esters of fatty acid and of sucrose, of maltose, of glucose or of fructose, mention may be made of sucrose monostearate, sucrose distearate and sucrose tristearate and mixtures thereof, such as the products sold by the company Croda under the name Crodesta F50, F70, F110 and F160; and examples of esters or mixtures of esters of fatty acid and of methylglucose which may be mentioned are methylglucose polyglyceryl-3 distearate, sold by the company Goldschmidt under the name Tego-care 450. Mention may also be made of glucose or maltose monoesters such as methyl o-hexadecanoyl-6-D-glucoside and o-hexadecanoyl-6-D-maltoside.

The (3) fatty alcohol ethers of sugars, which can be used as the above nonionic surfactant, may be solid at a temperature of less than or equal to 45° C. and may be chosen in particular from the group comprising ethers or mixtures of ethers of C₈-C₂₂ fatty alcohol and of glucose, of maltose, of sucrose or of fructose, and ethers or mixtures of ethers of a C₁₄-C₂₂ fatty alcohol and of methylglucose. These are in particular alkylpolyglucosides.

The C₈-C₂₂ or C₁₄-C₂₂ fatty alcohols forming the fatty unit of the ethers which may be used in the nanoemulsion of the invention comprise a saturated or unsaturated, linear alkyl or alkenyl chain containing, respectively, from 8 to 22 or from 14 to 22 carbon atoms. The fatty unit of the ethers may be chosen in particular from decyl, cetyl, behenyl, arachidyl, stearyl, palmityl, myristyl, lauryl, capryl and hexadecanoyl units, and mixtures thereof, such as cetearyl.

As examples of fatty alcohol ethers of sugars, mention may be made of alkylpolyglucosides such as decylglucoside and laurylglucoside, which is sold, for example, by the company Henkel under the respective names Plantaren 2000 and Plantaren 1200, cetostearyl glucoside optionally as a mixture with cetostearyl alcohol, sold for example, under the name Montanov 68 by the company SEPPIC, under the name Tego-care CG90 by the company Goldschmidt and under the name Emulgade KE3302 by the company Henkel, as well as arachidyl glucoside, for example in the form of a mixture of arachidyl alcohol and behenyl alcohol and arachidyl glucoside, sold under the name Montanov 202 by the company SEPPIC.

The surfactant used more particularly is sucrose monostearate, sucrose distearate or sucrose tristearate and mixtures thereof, methylglucose polyglyceryl-3 distearate and alkylpolyglucosides.

The (4) fatty esters of sorbitan and oxyalkylenated fatty esters of sorbitan which may be used as the above nonionic surfactant may be chosen from the group comprising C₁₆-C₂₂ fatty acid esters of sorbitan and oxyethylenated C₁₆-C₂₂ fatty acid esters of sorbitan. They may be formed from at least one fatty acid comprising at least one saturated linear alkyl chain containing, respectively, from 16 to 22 carbon atoms, and from sorbitol or from ethoxylated sorbitol. The oxyethylenated esters may generally comprise from 1 to 100 ethylene glycol units and preferably from 2 to 40 ethylene oxide (EO) units.

These esters may be chosen in particular from stearates, behenates, arachidates, palmitates, and mixtures thereof. Stearates and palmitates are preferably used.

As examples of the above nonionic surfactant can be used in the present invention, mention may be made of sorbitan monostearate (CTFA name: sorbitan stearate), sold by the company ICI under the name Span 60, sorbitan monopalmitate (CTFA name: sorbitan palmitate), sold by the company ICI under the name Span 40, and sorbitan tristearate 20 EO (CTFA name: polysorbate 65), sold by the company ICI under the name Tween 65.

The (4) oxyalkylenated fatty esters, preferably ethoxylated fatty esters, which may be used as the above nonionic surfactant, may be esters formed from 1 to 100 ethylene oxide units, preferably from 2 to 60 ethylene oxide units, and more preferably from 2 to 30 ethyl oxide units, and from at least one fatty acid chain containing from 8 to 22 carbon atoms, preferably from 8 to 18 carbon atoms, and more preferably from 8 to 12 carbon atoms. The fatty chain in the esters may be chosen in particular from stearate, behenate, arachidate and palmitate units, and mixtures thereof. Examples of ethoxylated fatty esters which may be mentioned are the ester of stearic acid comprising 40 ethylene oxide units, such as the product sold under the name Myrj 52 (CTFA name: PEG-40 stearate) by the company ICI, as well as the ester of behenic acid comprising 8 ethylene oxide units (CTFA name: PEG-8 behenate), such as the product sold under the name Compritol HD5 ATO by the company Gattefosse.

The (5) block copolymers of ethylene oxide (A) and of propylene oxide (B), which may be used as the above nonionic surfactant, may be chosen in particular from block copolymers of formula

HO(C₂H₄O)ₓ(C₃H₆O)_{y}(C₂H₄O)_{z}H (I)

in which x, y and z are integers such that x+z ranges from 2 to 100 and y ranges from 14 to 60, and mixtures thereof, and more particularly from the block copolymers of formula (I) having an HLB value ranging from 8.0 to 14.0.

The (6) polyoxyethylenated (1-40 EO) and polyoxypropylenated (1-30 PO) alkyl (C₁₆-C₃₀) ethers, which may be used as the above nonionic surfactant, may be selected from the group consisting of:

PPG-6 Decyltetradeceth-30; Polyoxyethlene (30) Polyoxypropylene (6) Tetradecyl Ether such as those sold as Nikkol PEN-4630 from Nikko Chemicals Co.,
PPG-6 Decyltetradeceth-12; Polyoxyethylene (12) Polyoxypropylene (6) Tetradecyl Ether such as those sold as Nikkol PEN-4612 from Nikko Chemicals Co.,
PPG-13 Decyltetradeceth-24; Polyoxyethylene (24) Polyoxypropylene (13) Decyltetradecyl Ether such as those sold as UNILUBE 50MT-2200B from NOF Corporation,
PPG-6 Decyltetradeceth-20; Polyoxyethylene (20) Polyoxypropylene (6) Decyltetradecyl Ether such as those sold as Nikkol PEN-4620 from Nikko Chemicals Co.,
PPG-4 Ceteth-1; Polyoxyethylene (1) Polyoxypropylene (4) Cetyl Ether such as those sold as Nikkol PBC-31 from Nikko Chemicals Co.,
PPG-8 Ceteth-1; Polyoxyethylene (1) Polyoxypropylene (8) Cetyl Ether such as those sold as Nikkol PBC-41 from Nikko Chemicals Co.,
PPG-4 Ceteth-10; Polyoxyethylene (10) Polyoxypropylene (4) Cetyl Ether such as those sold as Nikkol PBC-33 from Nikko Chemicals Co.,
PPG-4 Ceteth-20; Polyoxyethylene (20) Polyoxypropylene (4) Cetyl Ether such as those sold as Nikkol PBC-34 from Nikko Chemicals Co.,
PPG-5 Ceteth-20; Polyoxyethylene (20) Polyoxypropylene (5) Cetyl Ether such as those sold as Procetyl AWS from Croda Inc.,
PPG-8 Ceteth-20; Polyoxyethylene (20) Polyoxypropylene (8) Cetyl Ether such as those sold as Nikkol PBC-44 from Nikko Chemicals Co., and
PPG-23 Steareth-34; Polyoxyethylene Polyoxypropylene Stearyl Ether (34 EO) (23 PO) such as those sold as Unisafe 34S-23 from Pola Chemical Industries. They can provide a composition with stability for a long time, even though the temperature of the composition is increased and decreased in a relatively short period of time.

It may be more preferable that the polyoxyethylenated (1-40 EO) and polyoxypropylenated (1-30 PO) alkyl (C₁₆-C₃₀) ethers are (15-40 EO) and polyoxypropylenated (5-30 PO) alkyl (C₁₆-C₂₄) ethers, which could be selected from the group consisting of PPG-6 Decyltetradeceth-30, PPG-13 Decyltetradeceth-24, PPG-6 Decyltetradeceth-20, PPG-5 Ceteth-20, PPG-8 Ceteth-20, and PPG-23 Steareth-34.

It may be even more preferable that the polyoxyethylenated (1-40 EO) and polyoxypropylenated (1-30 PO) alkyl (C₁₆-C₃₀) ethers are (15-40 EO) and polyoxypropylenated (5-30 PO) alkyl (C₁₆-C₂₄) ethers, which could be selected from the group consisting of PPG-6 Decyltetradeceth-30, PPG-13 Decyltetradeceth-24, PPG-5 Ceteth-20, and PPG-8 Ceteth-20.

As (7) silicone surfactants, which may be used as the above nonionic surfactant, mention may be made of those disclosed in documents U.S. Pat. Nos. 5,364,633 and 5,411,744.

The (7) silicone surfactant as the above nonionic surfactant may preferably be a compound of formula (I):

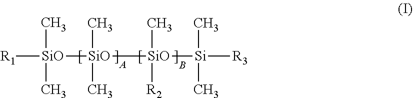

in which:
R₁, R₂ and R₃, independently of each other, represent a C₁-C₆ alkyl radical or a radical —(CH₂)ₓ—(OCH₂CH₂)_{y}—(OCH₂CH₂CH₂)_{z}—OR₄, at least one radical R₁, R₂ or R₃ not being an alkyl radical; R₄ being a hydrogen, an alkyl radical or an acyl radical;
A is an integer ranging from 0 to 200;
B is an integer ranging from 0 to 50; with the proviso that A and B are not simultaneously equal to zero;
x is an integer ranging from 1 to 6;
y is an integer ranging from 1 to 30;
z is an integer ranging from 0 to 5.

According to one preferred embodiment of the invention, in the compound of formula (I), the alkyl radical is a methyl radical, x is an integer ranging from 2 to 6 and y is an integer ranging from 4 to 30.

As examples of silicone surfactants of formula (I), mention may be made of the compounds of formula (II):

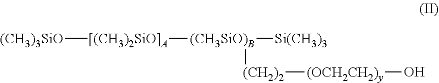

in which A is an integer ranging from 20 to 105, B is an integer ranging from 2 to 10 and y is an integer ranging from 10 to 20.

As examples of silicone surfactants of formula (I), mention may also be made of the compounds of formula (III):

H—(OCH₂CH₂)_{y}—(CH₂)₃[(CH₃)₂SiO]_{A′}—(CH₂)₃—(OCH₂CH₂)_{y}—OH (III)

in which A′ and y are integers ranging from 10 to 20.

Compounds of the invention which may be used are those sold by the company Dow Corning under the names DC 5329, DC 7439-146, DC 2-5695 and Q4-3667. The compounds DC 5329, DC 7439-146 and DC 2-5695 are compounds of formula (II) in which, respectively, A is 22, B is 2 and y is 12; A is 103, B is 10 and y is 12; A is 27, B is 3 and y is 12.

The compound Q4-3667 is a compound of formula (III) in which A is 15 and y is 13.

The amount of the (b) nonionic surfactant(s) in the composition according to the present invention may be 0.1% by weight or more, preferably 0.5% by weight or more, and more preferably 1% by weight or more relative to the total weight of the composition. It may be even more preferable that the amount of the (b) nonionic surfactant(s) in the composition according to the present invention be 1.5% by weight or more relative to the total weight of the composition.

On the other hand, the amount of the (b) nonionic surfactant(s) in the composition according to the present invention may be 30% by weight or less, preferably 20% by weight or less, and more preferably 10% by weight or less relative to the total weight of the composition. It may be even more preferable that the amount of the (b) nonionic surfactant(s) in the composition according to the present invention be 1.5% by weight or less relative to the total weight of the composition.

The amount of the (b) nonionic surfactant(s) in the composition according to the present invention may range from 0.1 to 30% by weight, preferably from 0.5 to 20% by weight, more preferably from 1 to 10% by weight relative to the total weight of the composition. It may be even more preferable that the amount of the (b) nonionic surfactant(s) in the composition according to the present invention be from 1.5% to 8% by weight relative to the total weight of the composition.

The weight ratio of the (b) nonionic surfactant(s) to the (a) ether oil(s) may be from 1:1 to 3:1, preferably from 1:1 to 2.67:1, and more preferably from 1:1 to 2:1.

### (Water)

The composition according to the present invention includes (c) water.

The amount of the (c) water in the composition according to the present invention is 70% by weight or more, preferably 80% by weight or more, and more preferably 90% by weight or more relative to the total weight of the composition.

On the other hand, the amount of the (c) water in the composition according to the present invention may be 99% by weight or less, preferably 98% by weight or less, and more preferably 97% by weight or less relative to the total weight of the composition.

The amount of (c) water in the composition according to the present invention may be from 70 to 99% by weight, preferably from 80 to 98% by weight, and more preferably from 90 to 97% by weight relative to the total weight of the composition.

### (Additional Oil)

The cosmetic composition according to the present invention may comprise (d) at least one additional oil other than the above (a) ether oil.

Here, “oil” means a fatty compound or substance which is in the form of a liquid or a paste (non-solid) at room temperature (25° C.) under atmospheric pressure (760 mmHg).

The (d) additional oil may be volatile or non-volatile, preferably volatile.

The (d) additional oil may be a non-polar oil such as a hydrocarbon oil; a polar oil such as a plant or animal oil and an ester oil; or a mixture thereof.

It may be preferable that the (a) additional oil be selected from non-polar oils, preferably hydrocarbon oils and more preferably volatile hydrocarbon oils.

As examples of plant oils, mention may be made of, for example, linseed oil, camellia oil, macadamia nut oil, corn oil, mink oil, olive oil, avocado oil, sasanqua oil, castor oil, safflower oil, jojoba oil, sunflower oil, almond oil, rapeseed oil, sesame oil, soybean oil, peanut oil, and mixtures thereof.

As examples of animal oils, mention may be made of, for example, squalene and squalane.

As examples of synthetic oils, mention may be made of ester oils and artificial triglyceride.

The ester oils are preferably liquid esters of saturated or unsaturated, linear or branched C₁-C₂₆ aliphatic monoacids or polyacids and of saturated or unsaturated, linear or branched C₁-C₂₆ aliphatic monoalcohols or polyalcohols, the total number of carbon atoms of the esters being greater than or equal to 10.

Preferably, for the esters of monoalcohols, at least one from among the alcohol and the acid from which the esters of the invention are derived is branched.

Among the monoesters of monoacids and of monoalcohols, mention may be made of ethyl palmitate, ethyl hexyl palmitate, ethyl hexyl myristate, isopropyl palmitate, isonononyl isononanoate, dicaprylyl carbonate, alkyl myristates such as isopropyl myristate, isopropyl palmitate, octyl isostearate or ethyl myristate, isocetyl stearate, 2-ethylhexyl isononanoate, isodecyl neopentanoate and isostearyl neopentanoate.

Esters of C₄-C₂₂ dicarboxylic or tricarboxylic acids and of C₁-C₂₂ alcohols and esters of monocarboxylic, dicarboxylic or tricarboxylic acids and of non-sugar C₄-C₂₆ dihydroxy, trihydroxy, tetrahydroxy or pentahydroxy alcohols may also be used.

Mention may especially be made of: diethyl sebacate; diisopropyl sebacate; bis(2-ethylhexyl) sebacate; diisopropyl adipate; di-n-propyl adipate; dioctyl adipate; bis(2-ethylhexyl) adipate; diisostearyl adipate; bis(2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; diethylene glycol diisononanoate.

As examples of preferable ester oils, mention may be made of, for example, diisopropyl adipate, dioctyl adipate, isononyl isononanoate, dicaprylyl carbonate, octyl isostearate, 2-ethylhexyl hexanoate, ethyl laurate, cetyl octanoate, octyldodecyl octanoate, isodecyl neopentanoate, myristyl propionate, 2-ethylhexyl 2-ethylhexanoate, 2-ethylhexyl octanoate, 2-ethylhexyl caprylate/caprate, methyl palmitate, ethyl palmitate, isopropyl palmitate, ethylhexyl palmitate, isohexyl laurate, hexyl laurate, isocetyl stearate, isopropyl isostearate, isopropyl myristate, isodecyl oleate, glyceryl tri(2-ethylhexanoate), pentaerythrityl tetra(2-ethylhexanoate), 2-ethylhexyl succinate, diethyl sebacate, and mixtures thereof.

As examples of artificial triglycerides, mention may be made of, for example, glyceryl trimyristate, glyceryl tripalmitate, glyceryl trilinolenate, glyceryl trilaurate, glyceryl tricaprate, glyceryl tricaprylate, glyceryl tri(caprate/caprylate) and glyceryl tri(caprate/caprylate/linolenate).

Hydrocarbon oils may be chosen from:
linear or branched, optionally cyclic, C₆-C₁₆ lower alkanes. Examples that may be mentioned include hexane, undecane, dodecane, tridecane, and isoparaffins, for instance isohexadecane, isododecane and isodecane; and
linear or branched hydrocarbons containing more than 16 carbon atoms, such as liquid paraffins, liquid petroleum jelly, polydecenes and hydrogenated polyisobutenes such as Parleam®, and squalane.

As preferable examples of hydrocarbon oils, mention may be made of, for example, linear or branched hydrocarbons such as mineral oil (e.g., liquid paraffin), paraffin, vaseline or petrolatum, naphthalenes, and the like; hydrogenated polyisobutene, isoeicosan, and decene/butene copolymer; and mixtures thereof.

It is preferable that the (d) additional oil be chosen from hydrocarbon oils which are in the form of a liquid at a room temperature.

It is also preferable that the (d) additional oil be chosen from oils with molecular weight below 600 g/mol.

Preferably, the (d) additional oil has a low molecular weight such as below 600 g/mol, chosen among ester oils with a short hydrocarbon chain or chains (e.g., isopropyl myristate, isopropyl palmitate, isononyl isononanoate, and ethyl hexyl palmitate), hydrocarbon oils with a short alkyl chain or chains (e.g., isododecane, isohexadecane, and squalane), short alcohol type oils such as octyldodecanol.

The amount of the (d) additional oil(s) in the composition according to the present invention may be 0.01% by weight or more, preferably 0.1% by weight or more, and more preferably 0.5% by weight or more relative to the total weight of the composition. It may be even more preferable that the amount of the (d) additional(s) in the composition according to the present invention be 1% by weight or more relative to the total weight of the composition.

On the other hand, the amount of the (d) additional oil(s) in the composition according to the present invention may be 20% by weight or less, preferably 10% by weight or less, and more preferably 5% by weight or less relative to the total weight of the composition. It may be even more preferable that the amount of the (d) additional oil(s) in the composition according to the present invention be 3% by weight or less relative to the total weight of the composition.

The amount of the (d) additional oil(s) in the composition according to the present invention may range from 0.01 to 20% by weight, preferably from 0.1 to 10% by weight, more preferably from 0.5 to 5% by weight relative to the total weight of the composition. It may be even more preferable that the amount of the (d) additional oil(s) in the composition according to the present invention be from 1% to 3% by weight relative to the total weight of the composition.

The amount of the total amount of the (a) ether oil(s) and the (d) additional oil(s) in the composition according to the present invention may be 0.01% by weight or more, preferably 0.1% by weight or more, and more preferably 0.5% by weight or more relative to the total weight of the composition. It may be even more preferable that the total amount of the (a) ether oil(s) and the (d) additional oil(s) in the composition according to the present invention be 1% by weight or more relative to the total weight of the composition.

On the other hand, the total amount of the (a) ether oil(s) and the (d) additional oil(s) in the composition according to the present invention may be 20% by weight or less, preferably 10% by weight or less, and more preferably 5% by weight or less relative to the total weight of the composition. It may be even more preferable that the total amount of the (a) ether oil(s) and the (d) additional oil(s) in the composition according to the present invention be 3% by weight or less relative to the total weight of the composition.

The amount of the total amount of the (a) ether oil(s) and the (d) additional oil(s) in the composition according to the present invention may range from 0.01 to 20% by weight, preferably from 0.1 to 10% by weight, more preferably from 0.5 to 5% by weight relative to the total weight of the composition. It may be even more preferable that the total amount of the (a) ether oil(s) and the (d) additional oil(s) in the composition according to the present invention be from 1% to 3% by weight relative to the total weight of the composition.

### (Optional Additives)

The composition according to the present invention may also comprise any optional additive(s) usually used in the field of cosmetics, chosen, for example, from anionic, cationic or amphoteric surfactants, solvents, gums, resins, hydrophilic thickening agents, hydrophobic thickening agents, dispersants, antioxidants, film-forming agents, preserving agents, fragrances, neutralizers, pH adjusting agents, antiseptics, UV-screening agents, cosmetic active agents such as vitamins, moisturizers, emollients or collagen-protecting agents, and mixtures thereof.

As the pH adjusting agent, at least one acidifying agent and/or at least one basifying agent (alkaline agent) may be used.

The acidifying agents can be, for example, mineral or organic acids, for instance hydrochloric acid, phosphoric acid, carboxylic acids, for instance tartaric acid, citric acid, and lactic acid, or sulphonic acids.

The acidifying agent may be present in an amount ranging from less than 5% by weight, preferably from 3% by weight or less, and more preferably from 1% by weight or less relative to the total weight of the composition.

The basifying agent or alkaline agent can be, for example, any inorganic or organic basic agents which are commonly used in cosmetic products such as ammonia; alkanolamines such as mono-, di- and tri-ethanolamine, isopropanolamine; sodium and potassium hydroxides; urea, guanidine and their derivatives; basic amino acids such as lysine or arginine; and diamines such as those described in the structure below:

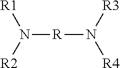

wherein

R denotes an alkylene such as propylene optionally substituted by a hydroxyl or a C₁-C₄ alkyl radical, and R₁, R₂, R₃, and R₄ independently denote a hydrogen atom, an alkyl radical, or a C₁-C₄ hydroxyalkyl radical, which may be exemplified by 1,3-propanediamine, and derivatives thereof. Arginine, urea, and monoethanolamine may be preferable.

The composition according to the present invention may comprise at least one water-miscible solvent such as a lower monoalcohol containing from 1 to 5 carbon atoms, C₃-C₄ ketones or C₃-C₄ aldehydes. The water-miscible solvent that can preferably be used is ethanol. The content of water-miscible solvent can range from 0.1% to 15% by weight, and better still from 1% to 8% by weight relative to the total weight of the composition.

It is a matter of routine operations for a person skilled in the art to adjust the nature and amount of the above optional additives which may be present in the composition in accordance with the present invention such that the desired cosmetic properties are not thereby affected.

### [Preparation and Properties]

The composition according to the present invention can be prepared by mixing the above-described essential and optional ingredients in a conventional manner.

For example, the composition according to the present invention can be prepared by a process comprising the step of mixing
(a) at least one ether oil;
(b) at least one nonionic surfactant; and
(c) water,
   such that
   the amount of the (c) water in the composition is 70% by weight or more, preferably 80% by weight or more, and more preferably 90% by weight or more relative to the total weight of the composition, and
   the oil droplet size in the composition is less than 100 nm, preferably less than 90 nm, and more preferably less than 80 nm.

It is possible to further mix any of the optional ingredients.

The composition according to the present invention may be in the form of a nano- or micro-emulsion.

The “micro-emulsion” may be defined in two ways, namely, in a broader sense and in a narrower sense. That is to say, there are one case (“microemulsion in the narrow sense”) in which the microemulsion refers to a thermodynamically stable isotropic single liquid phase containing a ternary system having three ingredients of an oily component, an aqueous component and a surfactant, and the other case (“micro-emulsion in the broad sense”) in which among thermodynamically unstable typical emulsion systems the microemulsion additionally includes those such emulsions presenting transparent or translucent appearances due to their smaller particle sizes (Satoshi Tomomasa, et al., OilChemistry, Vol. 37, No. 11 (1988), pp. 48-53).

The micro-emulsion refers to either one state of an O/W (oil-in-water) type microemulsion in which oil is solubilized by micelles, a W/O (water-in-oil) type microemulsion in which water is solubilized by reverse micelles, or a bicontinuous microemulsion in which the number of associations of surfactant molecules are rendered infinite so that both the aqueous phase and oil phase have a continuous structure.

The “nano-emulsion” here means an emulsion characterized by a dispersed phase with a size of less than 100 nm, the dispersed phase being stabilized by a crown of the (b) nonionic surfactant and the like that may optionally form a liquid crystal phase of lamellar type, at the dispersed phase/continuous phase interface. In the absence of specific opacifiers, the transparency of the nano-emulsions arises from the small size of the dispersed phase, this small size being possibly obtained by virtue of the use of mechanical energy and especially a high-pressure homogenizer.

Nanoemulsions can be distinguished from microemulsions by their structure. Specifically, micro-emulsions are thermodynamically stable dispersions formed from, for example, micells which are formed by the (b) nonionic surfactant and the like, and are swollen with the (a) ether oil and (d) additional oil, if present. Furthermore, microemulsions do not require substantial mechanical energy in order to be prepared.

The composition according to the present invention is in the form of an O/W nano- or micro-emulsion.

It is preferable that the composition according to the present invention be in the form of an O/W nano- or micro-emulsion wherein the oil phase in the form of an oil droplet has a volume-average particle size of 100 nm or less, preferably less than 90 nm, and more preferably less than 80 nm, for example, from 10 to 95 nm, preferably from 10 to 85 nm and more preferably from 10 to 70 nm.

The composition according to the present invention can have a transparent or slightly translucent appearance, preferably a transparent appearacnce. The transparent appearance of the composition according to the present invention may have a slight blue color.

The measurement of the appearance may be taken on the undiluted composition. The blank is determined with distilled water. The transparency may be measured by measuring the nephelometric turbidity (for example, with 2100Q Portable Turbidimeter from HACH).

The composition according to the present invention may preferably have a nephelometric turbidity lower than 150 NTU, preferably lower than 100 NTU, and more preferably lower than 50 NTU.

### [Cosmetic Composition]

The composition according to the present invention may preferably be used as a cosmetic composition. Thus, the composition according to the present invention may be intended for application onto a keratin substance. Keratin substance here means a material containing keratin as a main constituent element, and examples thereof include the skin, scalp, nails, lips, hair, eyelashes, eyeblow, and the like. Thus, it is preferable that the composition according to the present invention be used for a cosmetic process for the keratin substance.

It is preferable that the composition according to the present invention be a cleansing composition, more preferably a makeup remover, in particular a makeup remover from the keratin substance.

It is preferable that the composition according to the present invention has good fluidity while it has enhanced viscosity such that the composition does not drip off from the keratin substance.

The viscosity of the composition according to the present invention is not particularly limited, as long as the composition according to the present invention is fluidable while it does not drip off from the keratin substance. The viscosity can be measured at 25° C. with viscosimeters or rheometers preferably with cone-plane geometry. Preferably, the viscosity of the composition according to the present invention can range, for example, from 1 to 2000 Pa·s, and preferably from 1 to 1000 Pa·s at 25° C. and 1 s⁻¹.

### [Process and Use]

The composition according to the present invention can be used for a non-therapeutic process, such as a cosmetic process, for treating a keratin substance such as skin, hair, mucous membranes, nails, eyelashes, eyebrows, and scalp, by being applied to the keratin substance.

It is preferable that the process according to the present invention is a cleansing process, in particular, for washing out make-up products such as mascaras from the keratin substance.

The composition according to the present invention can be used, as it is, or in a cosmetic product, preferably a cleansing product. In particular, the composition according to the present invention may preferably be a cleansing product such as a make-up removing product for the body and/or facial skin, or keratin fibers such as hair, eyelashes, and eyebrow. Alternatively, the composition according to the present invention can be used as an element of the above product. For example the composition according to the present invention can be added to or combined with any other elements to form the above product.

The present invention also relates to a use of (a) at least one ether oil in a composition, in the form of an oil-in-water emulsion, comprising (b) at least one nonionic surfactant and (c) water, wherein the amount of the (c) water in the composition is 70% by weight or more, preferably 80% by weight or more, and more preferably 90% by weight or more relative to the total weight of the composition,

in order to make the oil droplet size in the composition less than 100 nm, preferably less than 90 nm, and more preferably less than 80 nm, and/or
in order to enhance the cleansing ability of the composition.

The explanations regarding the (a) ether oil(s), the (b) nonionic surfactant(s), the (c) water for the composition according to the present invention can apply to the process and use according to the present invention.

The composition used in the process and use according to the composition may include any of the optional ingredients as explained above for the composition according to the present invention.

### EXAMPLES

The present invention will be described in more detail by way of examples which however should not be construed as limiting the scope of the present invention.

### Examples A-F

The following compositions according to Examples A-F, shown in Table 1, were prepared by mixing the components shown in Table 1. The numerical values for the amounts of the components shown in Table 1 are all based on “% by weight” as active raw materials.

TABLE 1

| | Ex- | Ex- | Ex- | Ex- | Ex- | Ex- |
|---|---|---|---|---|---|---|
| | ample A | ample B | ample C | ample D | ample E | ample F |
| Polyglyceryl-2 | — | 2 | 2 | — | — | — |
| Caprate | | | | | | |
| Polyglyceryl-4 | 0.67 | — | — | — | 2 | — |
| Caprate | | | | | | |
| Polyglyceryl-6 | — | — | — | 3 | 2 | 3 |
| Dicaprate | | | | | | |
| Polyglyceryl-6 | — | — | — | — | — | 3 |
| Caprylate | | | | | | |
| Laureth-4 | 1.33 | 1 | 1 | — | — | — |
| PEG6 | — | — | — | 3 | — | — |
| Caprylic/Capric | | | | | | |
| Glycerides | | | | | | |
| PEG7 | 0.67 | — | — | — | — | — |
| Caprylic/Capric | | | | | | |
| Glycerides | | | | | | |
| Dicaprylyl | 1 | 1.5 | 3 | 3 | 3 | 3 |
| Ether | | | | | | |
| Isohexadecane | — | 1.5 | — | — | — | — |
| Preservatives | 0.07 | 0.27 | 0.27 | 0.32 | 0.47 | 0.32 |
| Water | qsp 100 | qsp 100 | qsp 100 | qsp 100 | qsp 100 | qsp 100 |
| Volume | 16.5 | 56.2 | 41.8 | 16.0 | 29.0 | 67.6 |
| Average | | | | | | |
| Particle | | | | | | |
| Diameter (nm) | | | | | | |

### Comparative Examples 1-4

The following compositions according to Examples 1-4, shown in Table 2, were prepared by mixing the components shown in Table 2. The numerical values for the amounts of the components shown in Table 2 are all based on “% by weight” as active raw materials.

TABLE 2

| | Comp. | Comp. | Comp. | Comp. |
|---|---|---|---|---|
| | Ex. 1 | Ex. 2 | Ex. 3 | Ex. 4 |
| Compared to | Ex. C | Ex. D | Ex. E | Ex. F |
| Polyglyceryl-2 Caprate | 2 | — | — | — |
| Polyglyceryl-4 Caprate | — | — | 2 | — |
| Polyglyceryl-6 Dicaprate | — | 3 | 2 | 3 |
| Polyglyceryl-6 Caprylate | — | — | — | 3 |
| Laureth-4 | 1 | — | — | — |
| PEG6 | — | 3 | — | — |
| Caprylic/Capric Glycerides | | | | |
| Ethylhexylpalmitate | 3 | 3 | 3 | 3 |
| Preservatives | 0.27 | 0.32 | 0.47 | 0.32 |
| Water | qsp 100 | qsp 100 | qsp 100 | qsp 100 |
| Volume Average Particle | 1305.5 | 1922.2 | 1922.2 | 1922.2 |
| Diameter (nm) | | | | |

### [Evaluation]

The volume-average particle diameter, transparency, and makeup removability of each of the compositions according to Examples A-F and Comparative Examples 1-4 were evaluated as follows.

### (Volume Average Particle Diameter)

The volume average particle diameter of the dispersed phase in each of the compositions according to Examples A-E and Comparative Examples 1-4 was measured with ELSZ-2000 zeta-potential & particle size analyzer from Otsuka Electronics.

The results are shown in Tables 1 and 2.

### (Transparency)

The transparency of each of the compositions according to Examples C-F and Comparative Examples 1-4 was evaluated by visual observation in accordance with the following criteria.

### Excellent: Transparent

Good: Slightly translucent
Poor: Opaque (cloudy)

The results are shown in Table 3.

TABLE 3

| | Comp. | | Comp. | | Comp. | | Comp. |
|---|---|---|---|---|---|---|---|
| Ex. C | Ex. 1 | Ex. D | Ex. 2 | Ex. E | Ex. 3 | Ex. F | Ex. 4 |
| Good | Poor | Excellent | Poor | Excellent | Poor | Good | Poor |

### (Makeup Removability)

The same amount of a mascara product (Maybelline NY Magnum Push-up Water-Proof mascara) was applied in the same manner onto fake eyelashes of each of test samples with the fake eyelashes.

The same amount of each of the compositions according to Examples A to F and Comparative Examples 1-4 was applied in the same manner onto each of cotton pieces with the same size and weight.

Then, the cotton piece was placed on the test sample, and moved back and forth on the test sample with the same speed, applied force, moving distance and repeating time. The amount of the composition removed from the test sample was measured by subtracting the weight of the cotton piece before use from the weight of the cotton piece after use. Thus, the makeup removability was determined by the following formula:

Makeup Removability (%)=(the weight of the removed composition)/(the weight of the applied composition)*100

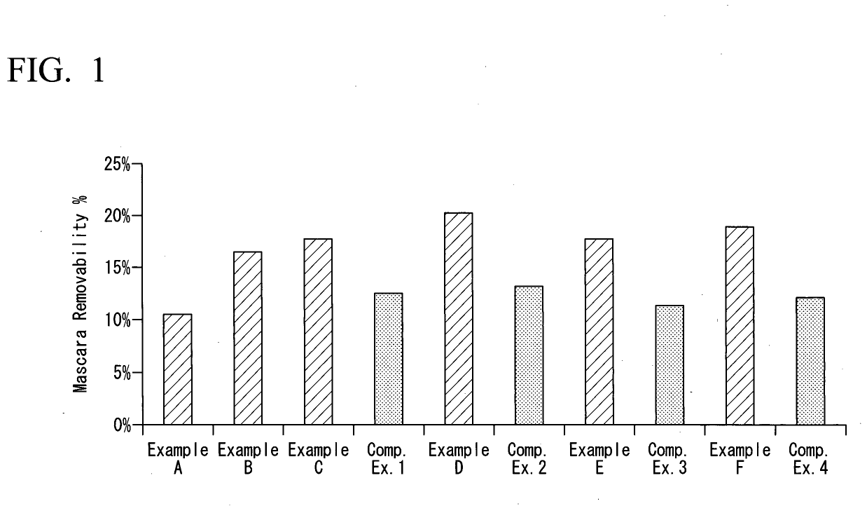

The results are shown in FIG. 1.

Examples A-F show that the compositions according to the present invention can be transparent or slightly translucent, and can have a good cleansing ability. The compositions according to Examples A-F include a substantial amount of water, and therefore, they can provide good feeling on the skin after use, such as refreshing, smoothing and moisturizing sensation.

Comparative Examples 1-4 show that the compositions according to Comparative Examples 1-4 which include ethylhexylpalmitate instead of the (a) ether oil cannot be transparent or slightly translucent, and cannot have better cleansing ability than the compositions according to Examples C-F in terms of the formulation of the compositions. It should be noted that Comparative Examples 1-4 should be compared with Example C-F.

In view of Examples D and F, it is preferable that the composition according to the present invention include, as the (b) nonionic surfactant, a mixture of polyglyceryl fatty acid ester and polyoxyethylenated alkyl glycerides.

1. A composition in the form of an oil-in-water emulsion, comprising:
(a) at least one ether oil;
(b) at least one nonionic surfactant; and
(c) water,
wherein the amount of the (c) water in the composition is 70% by weight or more relative to the total weight of the composition, and
the oil droplet size in the composition is less than 100 nm.

2. The composition according to claim 1, wherein the (a) ether oil is selected from the group consisting of dicaprylyl ether, dicapryl ether, dilauryl ether, diisostearyl ether, dioctyl ether, nonyl phenyl ether, dodecyl dimethylbutyl ether, cetyl dimethylbutyl ether, cetyl isobutyl ether, and mixtures thereof.

3. The composition according to claim 1, wherein the amount of the (a) ether oil(s) in the composition ranges from 0.01 to 20% by weight relative to the total weight of the composition.

4. The composition according to claim 1, wherein the (b) nonionic surfactant has an HLB value of from 8.0 to 14.0.

5. The composition according to claim 1, wherein the (b) nonionic surfactant is chosen from:
(1) surfactants chosen from polyglyceryl fatty acid esters, polyoxyalkylenated alkyl glycerides, and polyoxyalkylenated fatty ethers;
(2) mixed esters of fatty acid or of fatty alcohol, of carboxylic acid and of glycerol;
(3) fatty acid esters of sugars and fatty alcohol ethers of sugars;
(4) surfactants chosen from fatty esters of sorbitan and oxyalkylenated fatty esters of sorbitan, and oxyalkylenated fatty esters;
(5) block copolymers of ethylene oxide (A) and of propylene oxide (B),
(6) polyoxyethylenated (1-40 EO) and polyoxypropylenated (1-30 PO) alkyl (C₁₆-C₃₀) ethers,
(7) silicone surfactants, and
(8) mixtures thereof.

6. The composition according to claim 1, wherein the (b) nonionic surfactant is chosen from:
polyglyceryl fatty acid esters of at least one fatty acid comprising at least one saturated or unsaturated, linear or branched C₈-C₂₂ hydrocarbon group and of 2-12 glycerols;
polyoxyethylenated alkyl glycerides;
polyoxyethylenated fatty ethers of at least one fatty alcohol comprising at least one saturated or unsaturated, linear or branched C₈-C₂₂ hydrocarbon group, and of 2 to 60 ethylene oxides; and
mixtures thereof.

7. The composition according to claim 1, wherein the amount of the (b) nonionic surfactant(s) in the composition ranges from 0.1 to 30% by weight relative to the total weight of the composition.

8. The composition according to claim 1, wherein the weight ratio of the (b) nonionic surfactant(s) to the (a) ether oil(s) is from 1:1 to 3:1.

9. The composition according to claim 1, further comprising (d) at least one additional oil.

10. The composition according to claim 9, wherein the (d) additional oil is selected from non-polar oils.

11. The composition according to claim 9, wherein the amount of the (d) additional oil(s) in the composition ranges from 0.01 to 20% by weight relative to the total weight of the composition.

12. The composition according to claim 9, wherein the total amount of the (a) ether oil(s) and the (d) additional oil(s) in the composition ranges from 0.01 to 20% by weight relative to the total weight of the composition.

13. The composition according to claim 1, which is used as it is or used for a cleansing product.

14. A process for cleansing a keratin substance, comprising applying the composition according to claim 1 to the keratin substance.

15. A process for preparing a composition, comprising:
including (a) at least one ether oil in a composition, in the form of an oil-in-water emulsion, comprising (b) at least one nonionic surfactant and (c) water,
wherein the amount of the (c) water in the composition is 70% by weight or more relative to the total weight of the composition, and
at least one of following conditions is met:
(i) an oil droplet size in the composition is less than 100 nm, or
(ii) the (a) ether oil(s) enhance the cleansing ability of the composition.
